(19) United States
(12) Patent Application Publication
(10) Pub. No.: US 2023/0346765 A1
(43) Pub. Date: Nov. 2, 2023

(54) METHODS OF TREATING XANTHINE OXIDASE-RELATED DISEASES WITH NIFLUMIC ACID AND DERIVATIVES THEREOF

(71) Applicant: Baylor College of Medicine, Houston, TX (US)

(72) Inventors: Changyi Chen, Houston, TX (US); Qizhi Yao, Houston, TX (US); Jian-Ming Lu, Houston, TX (US)

(73) Assignee: Baylor College of Medicine, Houston, TX (US)

(21) Appl. No.: 17/905,995

(22) PCT Filed: Mar. 3, 2021
(86) PCT No.: PCT/US2021/020689

§ 371 Date: Sep. 9, 2022

Related U.S. Application Data
(60) Provisional application No. 62/991,830, filed on Mar. 19, 2020.

(51) Int. Cl. A61K 31/455; A61K 31/5377; A61K 31/519; A61K 31/11; A61P 19/06

(52) U.S. Cl. CPC A61K 31/455; A61K 31/5377; A61K 31/519; A61K 31/11; A61P 19/06

(57) ABSTRACT

Provided herein are methods of using niflumic acid and derivatives thereof for treating a disease or condition associated with elevated serum uric levels in a subject. The methods include administering to the subject an effective amount of a pharmaceutical composition as described herein. The disease or condition associated with elevated serum uric levels can include, for example, gout, hyperuricemia, or cardiovascular disease. Also provided herein are methods of reducing serum uric acid levels in a subject and methods for inhibiting xanthine oxidase activity in a cell. Novel pharmaceutical compositions are also provided herein. (I)

### CROSS-REFERENCE TO PRIORITY APPLICATION

This application claims priority to U.S. Provisional Application No. 62/991,830, filed Mar. 19, 2020, which is incorporated herein by reference in its entirety.

### BACKGROUND

The prevalence of gout and hyperuricemia in the United States is about 4% and 21%, respectively. The pooled prevalence of gout and hyperuricemia in mainland China from 2000 to 2014 was 1.1% and 13-25%, respectively. In the some areas of the world, the prevalence of gout and hyperuricemia is as high as up to 11.7% and 41.1%, respectively. A continuous increase in global prevalence of both hyperuricemia and chronic gout is expected as a consequence of lifestyle changes, increasing obesity, and ageing of the population.

Xanthine oxidase (XO) is a form of a molybdoflavin protein, xanthine oxidoreductase (XOR). It plays an important role in the catabolism of purines in humans, as it catalyzes the oxidation of hypoxanthine to xanthine and then catalyzes the oxidation of xanthine to uric acid. Meanwhile, reactive oxygen species (ROS), including superoxide and H₂O₂, are generated during this process. Uric acid can serve as an antioxidant to prevent macromolecular damage by ROS. However, overproduction of uric acid can cause hyperuricemia and lead to gout and other diseases. Therefore, maintaining uric acid at normal levels represents an important therapeutic goal for the prevention of gout and related disorders. For most patients with primary gout, overproduction of uric acid is the primary cause of hyperuricemia.

Currently, two drugs have been developed to treat gout. Allopurinol is the most commonly used therapy for chronic gout and has been used clinically for more than 40 years. Allopurinol lowers uric acid production by inhibiting XO activity, and is used as a first-line urate-lowering pharmacotherapy. Allopurinol, a structural isomer of hypoxanthine, is hydroxylated by XO to oxypurinol, which coordinates tightly to the reduced form of the molybdenum center, replacing the Mo—OH group of the native enzyme. Unfortunately, while rare, allopurinol has life-threatening side effects such as a hypersensitivity syndrome consisting of fever, skin rash, eosinophilia, hepatitis, and renal toxicity, for which the mortality rate approaches 20%. Allopurinol also causes Stevens-Johnson syndrome (SJS) and toxic epidertnal necrolysis (TEN), two life-threatening dermatological conditions. Febuxostat, a non-purine xanthine oxidase inhibitor, has been approved for the management of gout in Europe and the United States. Side effects associated with febuxostat therapy include elevated serum liver enzymes, nausea, diarrhea, arthralgia, headache, and rash. The drugs available for treatment and prevention of hyperuricemia and gout remain limited.

### SUMMARY

Provided herein are methods of treating a disease or condition associated with elevated serum uric acid levels in a subject, comprising administering to the subject an effective amount of a pharmaceutical composition comprising a compound of the following formula:

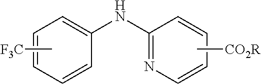

or a pharmaceutically acceptable salt thereof. In these compounds, R is hydrogen, substituted or unsubstituted alkyl, substituted or unsubstituted alkenyl, substituted or unsubstituted alkynyl, substituted or unsubstituted aryl, or substituted or unsubstituted cycloalkyl.

Optionally, the compounds for use in the methods described herein can be selected from the group consisting of:

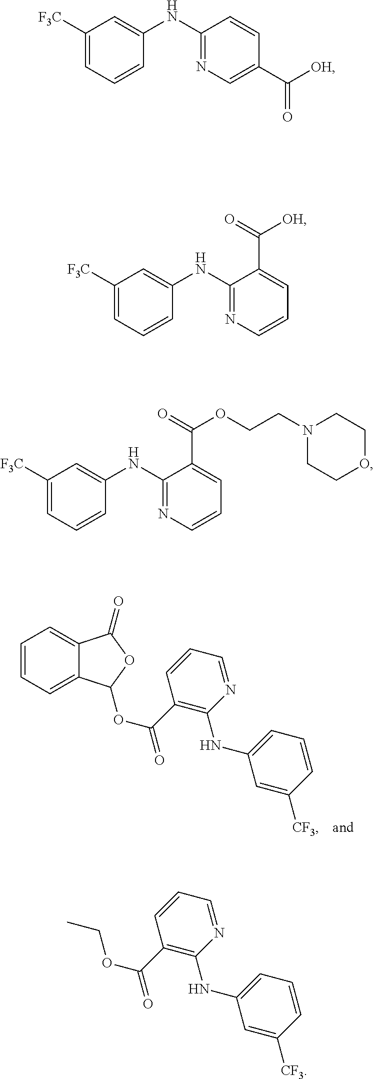 [SMILES: O=C(O)c1ccc(Nc2cccc(C(F)(F)F)c2)nc1 ; O=C(O)c1cccnc1Nc1cccc(C(F)(F)F)c1 ; O=C(OCCN1CCOCC1)c1cccnc1Nc1cccc(C(F)(F)F)c1 ; O=C1OC(OC(=O)c2cccnc2Nc2cccc(C(F)(F)F)c2)c2ccccc21 ; CCOC(=O)c1cccnc1Nc1cccc(C(F)(F)F)c1]

The disease or condition associated with elevated serum uric acid levels can include, for example, gout, hyperuricemia, or cardiovascular disease. The methods described herein can further include administering a second therapeutic agent to the subject. Optionally, the second therapeutic agent comprises allopurinol, folic acid, or 3,4-dihydroxy-5-nitrobenzaldehyde. Optionally, the methods described herein further comprise selecting a. subject having a disease or condition associated with elevated serum uric acid levels. The pharmaceutical compositions for use in the methods of treating a disease or condition associated. with elevated. serum uric acid levels in a subject as described herein can be substantially free from tetrazoles, triazoles, or solubility enhancers for monosodium urate.

Also provided herein are methods of reducing serum uric acid levels in a subject. The methods include administering to the subject an effective amount of a pharmaceutical composition comprising a compound as described herein. Optionally, the methods for reducing serum uric acid levels in a subject further comprise selecting a subject having a disease or condition associated with elevated serum uric acid levels (e.g., gout, hyperuricemia, or cardiovascular disease). The methods can further comprise administering a second therapeutic agent to the subject (e.g., allopurinol, folic acid, or 3,4-dihydroxy-5-nitrobenzaldehyde). The pharmaceutical compositions for use in the methods of reducing serum uric acid levels in a subject as described herein can be substantially free from tetrazoles, triazoles, or solubility enhancers for monosodium urate.

Further provided herein are methods of inhibiting xanthine oxidase activity in a cell. The methods include contacting a cell with an effective amount of a composition comprising a compound as described herein or a pharmaceutically acceptable salt thereof. Optionally, the methods for inhibiting xanthine oxidase activity in a cell further comprise contacting the cell with a second therapeutic agent (e.g., allopurinol, folic acid, or 3,4-dihydroxy-5-nitrobenzaldehyde). Optionally, the contacting is performed in vivo. Optionally, the contacting is performed in vitro. The compositions for use in the methods of inhibiting xanthine oxidase activity in a cell as described herein can be substantially free from tetrazoles, triazoles, or solubility enhancers for monosodium urate.

Also described herein are pharmaceutical compositions comprising

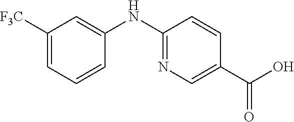 [SMILES: O=C(O)c1ccc(Nc2cccc(C(F)(F)F)c2)nc1]

and a pharmaceutically acceptable carrier. The pharmaceutical compositions described herein can further comprise a xanthine oxidase inhibitor (e.g., allopurinol). Optionally, the pharmaceutical compositions as described herein are substantially free from tetrazoles, triazoles, or solubility enhancers for monosodium urate.

The details of one or more embodiments are forth in the drawings and the description below. Other features, objects, and advantages will be apparent from the description and drawings, and from the claims.

### DESCRIPTION OF THE DRAWINGS

FIG. 1A is a graph showing the inhibition of xanthine oxidase (XO) activity by niflumic acid (NFA) in a cell free assay, FIG. 1B is a graph showing the influence of pre-incubation time of NFA with XO on the XO inhibitory activity by NFA. FIG. 1C is a graph showing the influence of pre-incubation of NFA with XO on the stability of NFA.

FIG. 2A is a graph of the consecutive formation of uric acid (295 nm arrow indicates increase) during the oxidation of xanthine by xanthine oxidase (XO). FIG. 2B is a graph of the consecutive formation of uric acid (295 nm arrow indicates increase) during the oxidation of xanthine by XO in the presence of niflumic acid (NFA).

FIG. 3A is a graph showing the inhibition kinetics of niflumic acid (NFA) on xanthine oxidase activity (XO). FIG. 3B is a plot of the data used to determine the enzyme kinetic parameters Vₘₐₓ and Kₘ for the response of XO to different concentrations of niflumic acid with a cell-free enzymatic assay. Vₘₐₓ is the maximum velocity of the enzyme and Kₘ is the Michaelis-Menten constant. FIG. 3C is a plot of the data used to determine the inhibition constant, Kᵢ of NFA for the inhibition of XO activity with a cell-free assay.

FIG. 4 is a graph comparing the inhibitory effects of niflumic acid (NFA) and other compounds on xanthine oxidase (XO) activity in a cell-free enzymatic assay. Each of the inhibitor compounds were at a concentration of 20 μM. The control sample represents no inhibitor added.

FIG. 5 is a graph demonstrating the dose-dependent effects of niflumic acid (NFA), NFA impurity E, flunixin, clonixin, and flufenamic acid on the inhibition of xanthine oxidase (XO) activity in a cell-free enzymatic assay. Allopurinol was used as a positive control and DMSO was used as a negative control.

FIG. 6 is a graph showing the influence of pre-incubation of niflumic acid (NFA) impurity E (0.35 μM) with 20 μM xanthine oxidase (XO).

FIG. 7A is a graph showing the inhibition kinetics of NFA impurity E on XO activity, FIG. 7B is a plot of the data used to determine the enzyme kinetic parameters Vₘₐₓ and Kₘ for the response of XO to different concentrations of NFA impurity E with a cell-free enzymatic assay. Vₘₐₓ is the maximum velocity of the enzyme and Kₘ is the Michaelis-Menten constant. FIG. 7C is a plot of the data used to determine the inhibition constant, Kᵢ of NFA impurity E for the inhibition of XO activity with a cell-free enzymatic activity.

FIG. 8A contains graphs showing the effects of combinations of 0.33 μM niflumic acid (NFA) and 0.67 μM allopurinol (left panel); 0.67 μM NFA and 1.33 μM allopurinol (right panel), on the inhibition of xanthine oxidase (XO) activity in a cell-free enzymatic assay. FIG. 8B is a graph showing the effect of a combination of 0.67 μM NFA and 0.67 μM 3,5-dihydroxy-5-nitrobenzaldehyde (DHNB) on the inhibition of XO activity in a cell-free enzymatic assay. FIG. 8C is a graph showing the effect of a combination of 0.67 μM NFA and 0.17 μM folic acid on the inhibition of XO activity in a cell-free enzymatic assay.

FIG. 9 contains graphs showing the effects of combinations of 0.67 μM allopurinol and 0.09 μM niflumic acid (NFA) impurity E (top left panel); 1.33 μM allopurinol and 0.09 μM NFA impurity E (top right panel); 1.33 μM allopurinol and 0.18 μM NFA impurity E (bottom left panel), on the inhibition of XO activity in a cell free enzymatic assay.

FIG. 10 is a graph showing the effects on serum uric levels in uricase −/− mice treated with niflumic acid (NFA), 3,5-dihydroxy-5-nitrobenzaldehyde (DHNB), and allopurinol in drinking water at different time points. Control mice received normal drinking water at all three time points.

FIG. 11 is a graph showing the effects on serum uric level in uricase mice treated with NFA, DHNB, and allopurinol in solid chow diet. Control mice received normal solid chow diet at all three time points.

FIG. 12 is a graph showing the effect on serum uric acid levels in allantoxanamide-treated mice treated with morniflumate. Positive control mice were treated with allopurinol at the same concentration as morniflumate and negative control mice were only treated with PEG400 following the initial treatment with allantoxanamide.

FIG. 13 is a graph showing the effects of 4 test compounds (including niflumic acid, niflumic acid (NFA) impurity E, talniflumate and ethylniflumate) on serum uric acid levels in allantoxanamide-treated mice.

### DETAILED DESCRIPTION

Provided herein are compounds and methods for their use in treating a disease or condition associated with elevated serum uric levels in a subject. The disease or condition associated with elevated serum uric levels comprises gout, hyperuricemia, or a cardiovascular disease. The compounds described herein are administered in an effective amount to a subject having a disease or condition associated with elevated serum uric acid levels.

### I. Compounds

A class of compounds described herein includes Formula I:

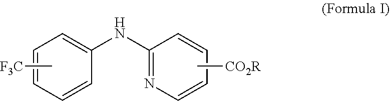

and pharmaceutically acceptable salts or prodrugs thereof.

In Formula 1, R is hydrogen, substituted or unsubstituted alk substituted or unsubstituted alkenyl, substituted or unsubstituted alkynyl, substituted or unsubstituted aryl, or substituted or unsubstituted cycloalkyl. Optionally, R is hydrogen. Optionally, R is substituted or unsubstituted alkyl (e.g., methyl, ethyl, propyl, or butyl). For example, R can be a heterocycloalkyl substituted-alkyl.

In some cases, the compounds according to Formula I are represented by Structure I-A:

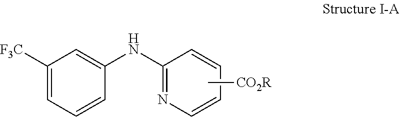

Examples of Structure I-A include the following compounds:

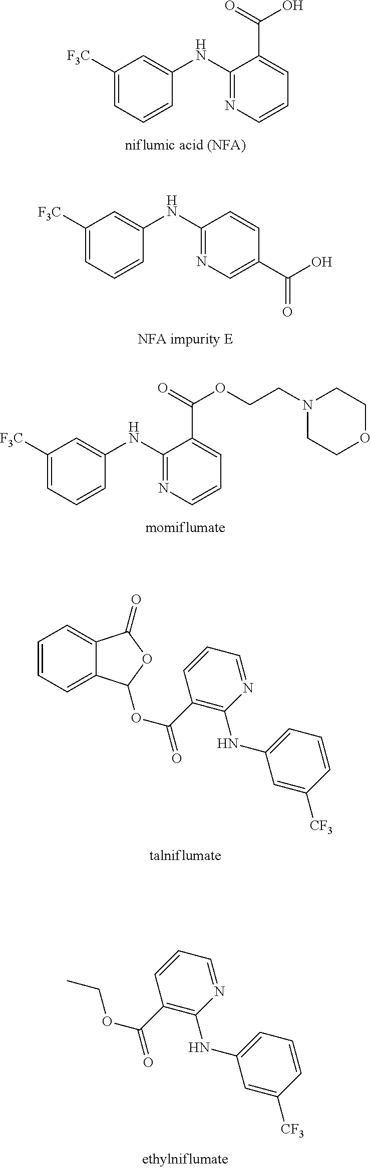 [SMILES: O=C(O)c1cccnc1Nc1cccc(C(F)(F)F)c1 ; O=C(O)c1ccc(Nc2cccc(C(F)(F)F)c2)nc1 ; O=C(OCCN1CCOCC1)c1cccnc1Nc1cccc(C(F)(F)F)c1 ; O=C1OC(OC(=O)c2cccnc2Nc2cccc(C(F)(F)F)c2)c2ccccc21 ; CCOC(=O)c1cccnc1Nc1cccc(C(F)(F)F)c1]

As used herein, the terms alkyl, alkenyl, and alkynyl include straight- and branched-chain monovalent substituents. Examples include methyl, ethyl, isobutyl, 3-butynyl, and the like. Ranges of these groups useful with the compounds and methods described herein include C₁-C₂₀ alkyl, C₂-C₂₀ alkenyl, and C₂-C₂₀ alkynyl. Additional ranges of these groups useful with the compounds and methods described herein include C₁-C₁₂ alkyl, C₂-C₁₂ alkenyl, C₂-C₁₂ alkynyl, C₁-C₆ alkyl, C₂-C₆ alkenyl, C₂-C₆ alkynyl, C₁-C₄ alkyl, C₂-C₄ alkenyl, and C₂-C₄ aknyl.

Heteroalkyl heteroalkenyl, and heteroalkynyl are defined similarly as alkyl, alkenyl, and alkynyl, but can contain O, S, or N heteroatoms or combinations thereof within the backbone. Ranges of these groups useful with the compounds and methods described herein include C₁-C₂₀ heteroalkyl, C₂-C₂₀ heteroalkenyl, and C₂-C₂₀ heteroalkynyl. Additional ranges of these groups useful with the compounds and methods described herein include C₁-C₁₂ heteroalkyl, C₂-C₁₂ heteroalkenyl, C₂-C₁₂heteroalkynyl, C₁-C₆ heteroalkyl, C₂-C₆ heteroalkenyl, C₂-C₆ heteroalkynyl, C₁-C₄ heteroalkyl, C₂-C₄ heteroalkenyl, and C₂-C₄ heteroalkynyl.

The terms cycloalkyl, cycloalkenyl, and cycloalkynyl include cyclic alkyl groups having a single cyclic ring or multiple condensed rings. Examples include cyclohexyl, cyclopentylethyl, and adamantanyl. Ranges of these groups useful with the compounds and methods described herein include C₃-C₂₀ cycloalkyl, C₃-C₂₀ cycloalkenyl, and C₃-C₂₀ cycloalkynyl. Additional ranges of these groups useful with the compounds and methods described herein include C₅-C₁₂ cycloalkyl, C₅-C₁₂ cycloalkenyl, C₅-C₁₂ cycloalkynyl, C₅-C₆ cycloalkyl, C₅-C₆ cycloalkenyl, and C₅-C₆ cycloalkynyl.

The terms heterocycloalkyl, heterocycloalkenyl, and heterocycloalkynyl are defined similarly as cycloalkyl, cycloalkenyl, and cycloalkynyl, but can contain O, S, or N heteroatoms or combinations thereof within the cyclic backbone. Ranges of these groups useful with the compounds and methods described herein include C₃-C₂₀ heterocycloalkyl, C₃-C₂₀ heterocycloalkenyl, and C₃-C₂₀ heterocycloalkynyl. Additional ranges of these groups useful with the compounds and methods described herein include C₅-C₁₂ heterocycloalkyl, C₅-C₁₂ heterocycloalkenyl, C₅-C₁₂ heterocycloalkynyl, C₅-C₆ heterocycloalkyl, C₅-C₆ heterocycloalkenyl, and C₅-C₆ heterocycloalkynyl.

Aryl molecules include, for example, cyclic hydrocarbons that incorporate one or more planar sets of, typically, six carbon atoms that are connected by delocalized electrons numbering the same as if they consisted of alternating single and double covalent bonds. An example of an aryl molecule is benzene. Heteroaryl molecules include substitutions along their main cyclic chain of atoms such as O, N, or S. When heteroatoms are introduced, a set of five atoms, e.g., four carbon and a heteroatom, can create an aromatic system. Examples of heteroaryl molecules include furan, pyrrole, thiophene, imadazole, oxazole, pyridine, and pyrazine. Aryl and heteroaryl molecules can also include additional fused rings, for example, benzofuran, indole, benzothiophene, naphthalene, anthracene, and quinoline. The aryl and heteroaryl molecules can be attached at any position on the ring, unless otherwise noted.

The term alkoxy as used herein is an alkyl group bonded through a single, terminal ether linkage. The term aryloxy as used herein is an arvi group bonded through a single, terminal ether linkage. Likewise, the terms alkenyloxy, alkyhyloxy, heteroalkyloxy, heteroalkenyloxy, heteroalkynyloxy, heteroaryloxy, cycloalkyloxy, and heterocycloalkyloxy as used herein are an alkenyloxy, alkynyloxy, heteroalkyloxy, heteroalkenyloxy, heteroaknyloxy, heteroaryloxy, cycloalkyloxy, and heterocycloalkyloxy group, respectively, bonded through a single, terminal ether linkage.

The term hydroxy as used herein is represented by the formula —OH.

The terms amine or amino as used herein are represented by the formula —NZ¹Z², where Z¹ and Z² can each he substitution group as described herein, such as hydrogen, an alkyl, halogenated alkyl, alkenyl, alkynyl, aryl, heteroaryl, cycloalkyl, cycloalkenyl, heterocycloalkyl, or heterocycloalkenyl group described above.

The alkoxy, aryloxy, amino, alkyl, alkenyl, alkynyl, aryl, heteroalkyl, heteroalkenyl, heteroalkynyl, heteroaryl, cycloalkyl, or heterocycloalkyl molecules used herein can be substituted or unsubstituted. As used herein, the term substituted includes the addition of an alkoxy, aryloxy, amino, alkyl, alkenyl, alkynyl, aryl, heteroalkyl, heteroalkenyl, heteroalkynyl, heteroaryl, cycloalkyl, or heterocycloalkyl group to a position attached to the main chain of the alkoxy, aryloxy, amino, alkyl, alkenyl, alkynyl, aryl, heteroalkyl, heteroalkenyl, heteroalkynyl, heteroaryl, cycloalkyl, or heterocycloalkyl, e.g., the replacement of a hydrogen by one of these molecules. Examples of substitution groups include, but are not limited to, hydroxy, halogen (e.g., F, Br, Cl, or I), and carboxyl groups. Conversely, as used herein, the term unsubstituted indicates the alkoxy, aryloxy, amino, alkyl, alkenyl, alkynyl, aryl, heteroalkyl, heteroalkenyl, heteroalkynyl, heteroaryl, cycloalkyl, or heterocycloalkyl has a full complement of hydrogens, i.e., commensurate with its saturation level, with no substitutions, e.g., linear decane (—(CH₂)₉—CH₃).

### II. Methods of Making the Compounds

The compounds described herein can be prepared in a variety of ways. The compounds can be synthesized using various synthetic methods. At least some of these methods are known in the art of synthetic organic chemistry. The compounds described herein can be prepared from readily available starting materials. Optimum reaction conditions may vary with the particular reactants or solvents used, but such conditions can he determined by one skilled in the art.

Variations on Formula I and the compounds described herein include the addition, subtraction, or movement of the various constituents as described for each compound. Similarly, when one or more chiral centers are present in a molecule, all possible chiral variants are included. Additionally, compound synthesis can involve the protection and deprotection of various chemical groups. The use of protection and deprotection, and the selection of appropriate protecting groups can be determined by one skilled in the art. The chemistry of protecting groups can be found, for example, in Wuts, Greene's Protective Groups in Organic Synthesis, 5th. Ed,, Wiley & Sons, 2014, which is incorporated herein by reference in its entirety.

Reactions to produce the compounds described herein can be carried out in solvents, which can be selected by one of ordinary skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H-NMR or ¹³C-NMR), infrared spectroscopy (IR), spectrophotometry (e.g., UV-visible), or mass spectrometry (MS), or by chromatography, such as high performance liquid chromatography (HPLC) or thin layer chromatography (TLC).

Optionally, the compounds described herein can be obtained from commercial sources. The compounds can be obtained from, for example, Sigma Chemical Co. (St. Louis, MO); VWR International (Radnor, PA); or Oakwood Products, Inc. (West Columbia, SC).

### III. Pharmaceutical Formulations

The compounds described herein or derivatives thereof can be provided in a pharmaceutical composition. Depending on the intended mode of administration, the pharmaceutical composition can be in the form of solid, semi-solid or liquid dosage forms, such as, for example, tablets, suppositories, pills, capsules, powders, liquids, or suspensions, preferably in unit dosage form suitable for single administration of a precise dosage. The compositions will include a therapeutically effective amount of the compound described herein or derivatives thereof in combination with a pharmaceutically acceptable carrier and, in addition, may include other medicinal agents, pharmaceutical agents, carriers, or diluents. By pharmaceutically acceptable is meant a material that is not biologically or otherwise undesirable, which can be administered to an individual along with the selected compound without causing unacceptable biological effects or interacting in a deleterious manner with the other components of the pharmaceutical composition in which it is contained.

The compositions can include one or more of the compounds described herein and a pharmaceutically acceptable carrier. As used herein, the term carrier encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington: The Science and Practice of Pharmacy, Adeboye Adejare ed., 23rd Ed., Academic Press (2021). Examples of physiologically acceptable carriers include buffers, such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers, such as polyvinylpyrrolidone; amino acids such as glycine, glutamine, asparagine, arginine or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN® (ICI, Inc.; Bridgewater, New Jersey), polyethylene glycol (PEG), and PLURONICS™ (BASF; Florham Park, NJ).

Compositions containing the compound described herein or derivatives thereof suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants, such as preserving, wetting, emulsifying, and dispensing agents. Prevention of the action of microorganisms can be promoted by various antibacterial and antifungal agents, for example, parabens, chlorobutanol, phenol, sorbic acid, and the like. Isotonic agents, for example, sugars, sodium chloride, and the like may also be included. Prolonged absorption of the injectable pharmaceutical form can he brought about by the use of agents delaying absorption, for example, aluminum monostearate and gelatin.

Solid dosage forms for oral administration of the compounds described herein or derivatives thereof include capsules, tablets, pills, powders, and granules. In such solid dosage forms, the compounds described herein or derivatives thereof is admixed with at least one inert customary excipient (or carrier), such as sodium citrate or dicalcium phosphate, or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others known in the art. They may contain opacifying agents and can also be of such composition that they release the active compound or compounds in a certain part of the intestinal tract in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration of the compounds described herein or derivatives thereof include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like.

Besides such inert diluents, the composition can also include additional agents, such as wetting, emulsifying, suspending, sweetening, flavoring, or perfuming agents.

Suspensions, in addition to the active compounds, may contain additional agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions of the compounds described herein or derivatives thereof for rectal administrations are optionally suppositories, which can be prepared by mixing the compounds with suitable non-irritating excipients or carriers, such as cocoa butter, polyethyleneglycol or a suppository wax, which are solid at ordinary temperatures but liquid at body temperature and, therefore, melt in the rectum or vaginal cavity and release the active component.

Dosage forms for topical administration of the compounds described herein or derivatives thereof include ointments, powders, sprays, and inhalants. The compounds described herein or derivatives thereof are admixed under sterile conditions with a physiologically acceptable carrier and any preservatives, buffers, or propellants as may be required. Ophthalmic formulations, ointments, powders, and solutions are also contemplated as being within the scope of the compositions.

As noted above, the compositions can include one or more of the compounds described herein or pharmaceutically acceptable salts thereof. As used herein, the term pharmaceutically acceptable salt refers to those salts of the compound described herein or derivatives thereof that are, within the scope of sound medical judgment, suitable for use in contact with the tissues of subjects without undue toxicity, irritation, allergic response, and the like, commensurate with a reasonable benefit/risk ratio, and effective for their intended use, as well as the zwitterionic forms, where possible, of the compounds described herein. The term salts refers to the relatively non-toxic, inorganic and organic acid addition salts of the compounds described herein. These salts can be prepared in situ during the isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, methane sulphonate, and laurylsulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See S. M. Barge et al., J. Pharm. Sci. (1977) 66, 1, which is incorporated herein by reference in its entirety, at least, for compositions taught therein.)

Optionally, the pharmaceutical compositions described herein can be substantially free from tetrazoles, triazoles, and/or solubility enhancers for monosodium urate. As used herein, the term “substantially free” from an indicated component (e.g., tetrazoles, triazoles, and/or solubility enhancers for monosodium urate), means that the pharmaceutical composition can include less than 1%, less than 0.1%, less than 0.01%, less than 0.001%, or less than 0.0001% of the component (e.g., a tetrazole, a triazole, and/or a solubility enhancer for monosodium urate) based on the weight of the pharmaceutical composition.

Optionally, the pharmaceutical compositions described herein can include a xanthine oxidase inhibitor. Optionally, the pharmaceutical compositions described herein can include anti-gout agents (e.g., allopurinol or 3,4-dihydroxy-5-nitrobenzaldehyde), anti-inflammatory agents, or antioxidants (e.g., folic acid).

Administration of the compounds and compositions described herein or pharmaceutically acceptable salts thereof can be carried out using therapeutically effective amounts of the compounds and compositions described herein or pharmaceutically acceptable salts thereof as described herein for periods of time effective to treat a disorder. The effective amount of the compounds and compositions described herein or pharmaceutically acceptable salts thereof as described herein may be determined by one of ordinary skill in the art and includes exemplary dosage amounts for a mammal of from about 0.5 to about 200 mg/g of body weight of active compound per day, which may be administered in a single dose or in the form of individual divided doses, such as from 1 to 4 times per day. Alternatively, the dosage amount can be from about 0.5 to about 150 mg/kg of body weight of active compound per day, about 0.5 to 100 mg/kg of body weight of active compound per day, about 0.5 to about 75 mg/kg of body weight of active compound per day, about 0.5 to about 50 mg/kg of body weight of active compound per day, about 0.01 to about 50 mg/kg of body weight of active compound per day, about 0.05 to about 25 mg/kg of body weight of active compound per day, about 0.1 to about 25 mg/kg of body weight of active compound per day, about 0.5 to about 25 mg/kg of body weight of active compound per day, about 1 to about 20 mg/kg of body weight of active compound per day, about 1 to about 10 mg/kg of body weight of active compound per day, about 20 mg/kg of body weight of active compound per day, about 10 mg/kg of body weight of active compound per day, about 5 mg/kg of body weight of active compound per day, about 2.5 mg/kg of body weight of active compound per day, about 1.0 mg/kg of body weight of active compound per day, or about 0.5 mg/kg of body weight of active compound per day, or any range derivable therein. Optionally, the dosage amounts are from about 0.01 mg/kg to about 10 mg/kg of body weight of active compound per day. Optionally, the dosage amount is from about 0.01 mg/kg to about 5 mg/kg. Optionally, the dosage amount is from about 0.01 mg/kg to about 2.5 mg/kg.

Those of skill in the art will understand that the specific dose level and frequency of dosage for any particular subject may be varied and will depend upon a variety of factors, including the activity of the specific compound employed, the metabolic stability and length of action of that compound, the species, age, body weight, general health, sex and diet of the subject, the mode and time of administration, rate of excretion, drug combination, and severity of the particular condition.

The precise dose to be employed in the formulation will also depend on the route of administration, and the seriousness of the disease or disorder, and should be decided according to the judgment of the practitioner and each subject's circumstances. Effective doses can be extrapolated from dose-response curves derived from in vitro or animal model test systems. Further, depending on the route of administration, one of skill in the art would know how to determine doses that result in a plasma concentration for a desired level of response in the cells, tissues and/or organs of a subject.

### IV. Methods of Use

Provided herein are methods of treating a disease or condition associated with elevated serum uric acid levels in a subject. Also described herein are methods of reducing uric acid production in a subject and for reducing serum uric acid levels in a subject. The methods include administering to a subject an effective amount of one or more of the compounds or pharmaceutical compositions described herein, or a pharmaceutically acceptable salt thereof. The expression “effective amount,” when used to describe an amount of compound in a method, refers to the amount of a compound that achieves the desired pharmacological effect or other effect, for example, an amount that results in decreased serum uric acid levels and/or decreased serum uric acid production.

The compounds and compositions described herein or pharmaceutically acceptable salts thereof are useful for treating and/or preventing a disease or condition associated with elevated serum uric acid levels including gout, hyperuricemia, or cardiovascular disease (e.g., abnormal heart rhythms; aortic aneurysms; cardiomyopathy; carditis; congenital heart disease; coronary artery diseases, such as angina, and myocardial infarction; heart failure; hypertensive heart disease; peripheral artery disease; rheumatic heart disease; stroke; thromboembolic disease; valvular heart disease; and venous thrombosis). In some cases, the compounds and compositions described herein are useful for treating and/or preventing diseases and conditions associated with hyperuricemia and/or xanthine-oxidase related diseases and conditions, such as arthritis, heart disease, hypertension, diabetes mellitus, vascular disease, stroke, erectile dysfunction, chronic kidney disease, immune and inflammatory diseases, psoriatic arthritis, osteroarthritis, micro-albuminuria, preeclampsia, cancer, nonalcoholic fatty liver disease (NAFLD), McArdle disease, chronic wounds, chronic obstructive pulmonary disease (COPD), Betcet's disease (BD), influenza A infection, restless legs syndrome, vitamin D insufficiency and/or deficiency, acute kidney injury, benign paroxysmal positional vertigo (BPPV), ocular abnormalities, pulmonary hypertension, hemolytic diseases, hypoxic-ischemic encephalopathy (HIE), and/or hemospermia.

The methods described herein are useful for treating the diseases and conditions described herein in humans, including, without limitation, pediatric and geriatric populations, and in animals, e.g., veterinary application. Optionally, the methods are used to treat conditions associated with elevated uric acid levels, including chronic gouty arthritis, acute inflammatory arthritis, uric acid nephropathy, kidney stones, or tophi. The methods can optionally include selecting a subject having a disease or condition associated with elevated serum uric acid levels. For example, the methods can include selecting a subject having gout, hyperuricemia, or cardiovascular disease.

The methods described herein can further comprise administering to the subject a second therapeutic agent. Thus, the provided compositions and methods can include one or more additional agents. The one or more additional agents and the compounds described herein or pharmaceutically acceptable salts thereof can be administered in any order, including concomitant, simultaneous, or sequential administration. Sequential administration can be temporally spaced order of up to several days apart. The methods can also include more than a single administration of the one or more additional agents and/or the compounds described herein or pharmaceutically acceptable salts or prodrugs thereof. The administration of the one or more additional agents and the compounds described herein or pharmaceutically acceptable salts or prodrugs thereof can be by the same or different routes and concurrently or sequentially.

Additional therapeutic agents include, but are not limited to, anti-gout agents. For example, the anti-gout agent can be allopurinol, 3,4-dihydroxy-5-nitrobenzaldehyde (DHNB), benzbromarone, colchicine, probenecid, or sulfinpyrazone. Therapeutic agents also include anti-inflammatory agents. Examples of suitable anti-inflammatory agents include, for example, steroidal and nonsteroidal anti-inflammatory drugs (e.g., ibuprofen and prednisone). The therapeutic agent can also be, for example, an antioxidant. Examples of suitable antioxidants include, for example, α-tocopherol, beta-carotene, butylated hydroxyanisole (BHA), butylated hdroxytoluene (BHT), caffeic acid, lutein, lycopene, selenium, tert-butylhydroquinone (TBHQ), Vitamin A, Vitamin B (folic acid), Vitamin C, and Vitamin E. Further examples of suitable antioxidants include putative antioxidant botanicals, such as, for example, grape seeds, green tea, Scutellaria baicalensis, American ginseng., ginkgo biloba, and the like.

Any of the aforementioned therapeutic agents can be used in any combination with the compositions described herein. Combinations are administered either concomitantly (e.g., as an admixture), separately but simultaneously (e.g., via separate intravenous lines into the same subject), or sequentially (e.g., one of the compounds or agents is given first followed by the second). Thus, the term combination is used to refer to concomitant, simultaneous, or sequential administration of two or more agents.

The methods and compounds as described herein are useful for both prophylactic and therapeutic treatment. For prophylactic use, a therapeutically effective amount of the compounds and compositions or pharmaceutically acceptable salts thereof as described herein are administered to a subject prior to onset (e.g., before obvious signs of gout, hyperuricemia, or cardiovascular disease), during early onset (e.g., upon initial signs and symptoms of gout, hyperuricemia, or cardiovascular disease), or after the development of gout, hyperuricemia, or cardiovascular disease. Prophylactic administration can occur for several days to years prior to the manifestation of symptoms of gout, hyperuricemia, or cardiovascular disease. Therapeutic treatment involves administering to a subject a therapeutically effective amount of the compounds and compositions or pharmaceutically acceptable salts thereof as described herein after gout, hyperuricemia, or cardiovascular disease is diagnosed.

The methods and compounds described herein are also useful in inhibiting xanthine oxidase activity in a cell. The methods include contacting a cell with an effective amount of a compound or composition as described herein. Optionally, the contacting is performed in vivo. Optionally, the contacting is performed in vitro.

### V. Kits

Also provided herein are kits for treating or preventing gout, hyperuricemia, or cardiovascular disease in a subject. A kit can include any of the compounds or compositions described herein. For example, a kit can include a compound of Formula I or any of the compounds described herein. A kit can further include one or more additional agents, such as anti-gout agents (e.g., allopurinol, 3,4-dihydroxy-5-nitrobenzaldehyde), anti-inflammatory agents, or antioxidants (e.g., folic acid). A kit can include an oral formulation of any of the compounds or compositions described herein. A kit can additionally include directions for use of the kit (e.g., instructions for treating a subject), a container, a means for administering the compounds or compositions, and/or a carrier.

As used herein the terms treatment, treat, or treating refer to a method of reducing one or more symptoms of a. disease or condition. Thus in the disclosed method, treatment can refer to a 10%, 20%, 30%. 40%, 50%, 60%, 70%, 80%, 90%, or 100% reduction in the severity of one or more symptoms of the disease or condition. For example, a method for treating a disease is considered to be a treatment if there is a 10% reduction in one or more symptoms or signs of the disease in a subject as compared to a control. As used herein, control refers to the untreated condition, Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition.

As used herein, the terms prevent, preventing, and prevention of a disease or disorder refer to an action, for example, administration of a composition or therapeutic agent, that occurs before or at about the same time a subject begins to show one or more symptoms of the disease or disorder, which inhibits or delays onset or severity of one or more symptoms of the disease or disorder.

As used herein, references to decreasing, reducing, or inhibiting include a change of 10%, 20%, 30%, 40%, 50%, 60%, 70%, 80%, 90% or greater as compared to a control level, Such terms can include, but do not necessarily include, complete elimination.

As used herein, subject means both mammals and non-mammals. Mammals include, for example, humans; non-human primates, e.g., apes and monkeys; cattle; horses; sheep; rats; mice; pigs; and goats. Non-mammals include, for example, fish and birds.

Throughout this application, various publications are referenced. The disclosures of these publications in their entireties are hereby incorporated by reference into this application.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the subject matter described herein which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

Xanthine oxidase from bovine milk, xanthine, allopurinol, niflumic acid (NFA), ethyl niflumate, 2-(3-chloro-5-(trifluoromethyl)-2-pyridylthio)pyridine-3-carboxylic acid, 2-[3-(trifluoromethyl)phenoxy]nicotinic acid, clonixin, flunixin, flufenamic acid, indomethacin, naproxen, diclofenac, aspirin, ibuprofen, talniflumate, 6-phenylamino-nicotinic acid, 2-phenylamino-nicotinic acid, N-phenylanthranilic acid, mefenamic acid, tolfenamic acid, meclofenamic acid, floctafenic acid, and dimethyl sulfoxide (DMSO) were obtained from Sigma Chemical Co. (St. Louis, MO). Morniflumate was purchased from Toronto Research Chemicals, Inc. (North York, Canada).

Data are presented as mean±SD as compared to the negative control. Statistical significance was determined by a Student's t-test (two tailed). A value of P<0.05 was considered significant.

### Example 1: XO Inhibition Assay

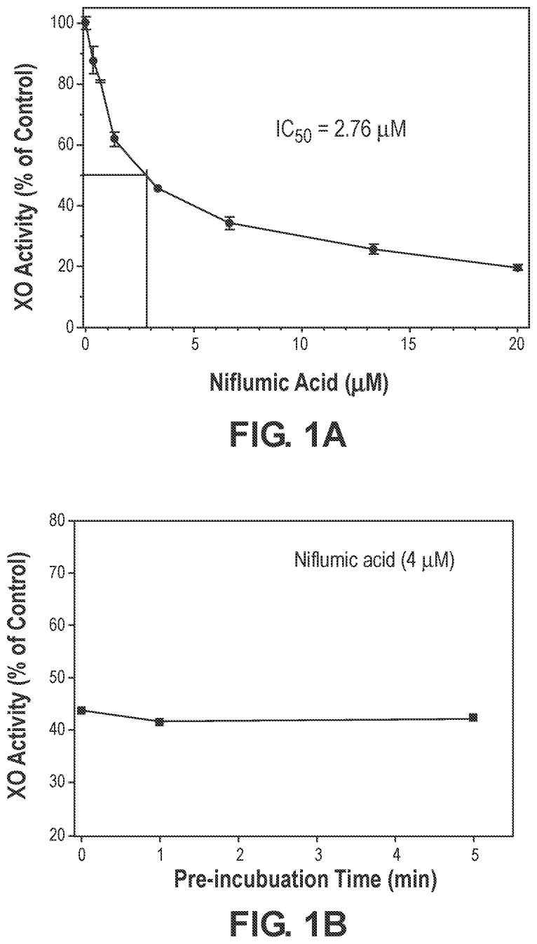

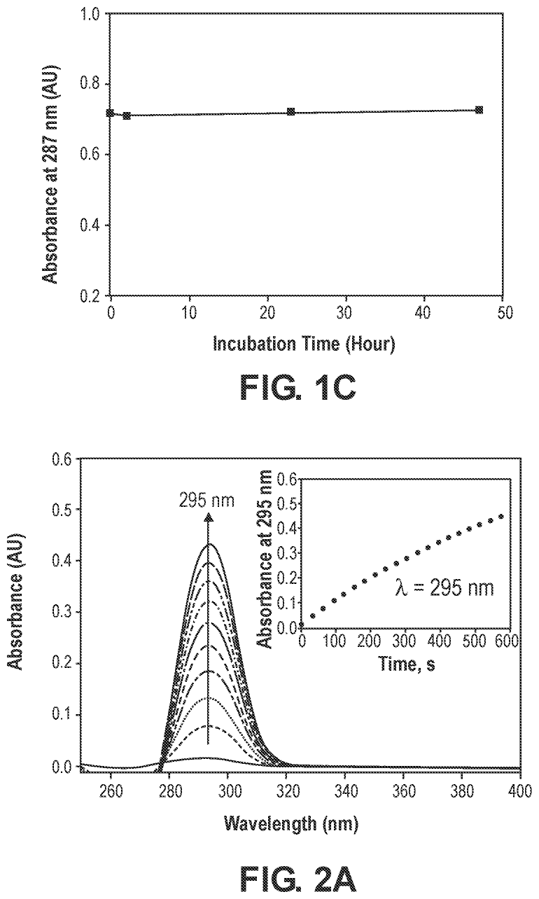

Niflumic acid (NFA) as a XO inhibitor—The inhibitory effect of niflumic acid (NFA) on xanthine oxidase (XO) was determined in vitro by spectrophotometrically monitoring the formation of uric acid. The uric acid absorbance at 295 nm was followed and the intensity data were collected every 30 seconds for 10 minutes. The initial rate of uric acid formation was generated for the reaction and converted to XO activity as 100% without any inhibitor (negative control). The inhibitory effect of NFA on XO activity was calculated as compared with the negative control (DMSO). When 20 nM XO was mixed with increasing concentrations of NFA, the initial rate of uric acid formation showed a concentration-dependent decrease compared to the control, reflecting the decrease of XO activity (see FIG. 1A). NFA showed a strong inhibitory effect on XO activity with a low IC₅₀ (2.75 μM). It exhibited strong inhibition at lower concentrations (≤3.3 μM) by decreasing XO relative activity to 45%. At 20 μM, it decreased the relative enzymatic activity to approximately 20% and exhibited a steady trend at high concentrations. When NFA was pre-incubated with XO for varying amounts of time followed by the addition of xanthine, the initial rate of uric acid formation did not change with longer pre-incubation times (see FIG. 1B). The stability of NFA towards pre-incubation times was established. An absorbance of NFA at 287 nm was collected after NFA was incubated with XO for 0 to 47 hours (see FIG. 1C), indicating pre-incubation had no effect on the stability of NFA.

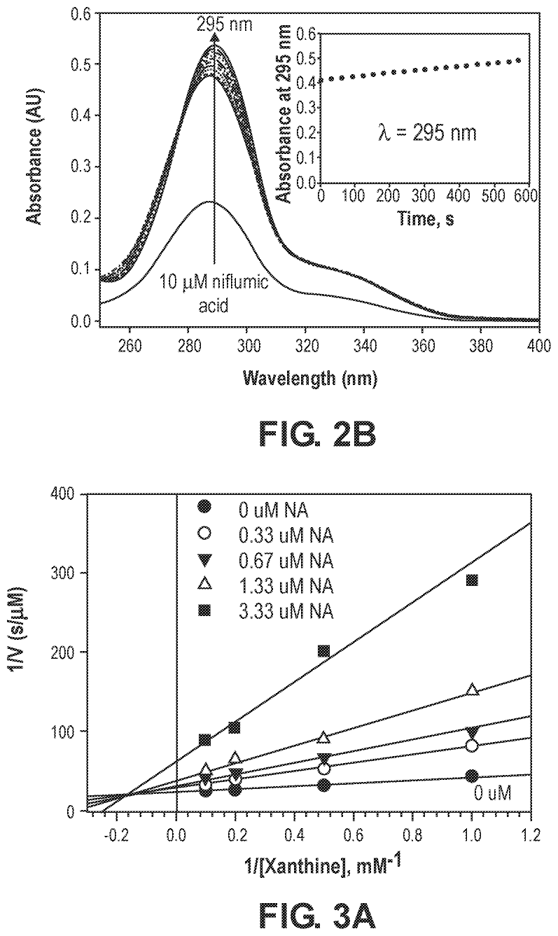

The formation of uric acid during the oxidation of xanthine by XO in the absence of NFA was recorded (see FIG. 2A). Uric acid formation was monitored as the absorbance at 295 nm over a period of 0-10 minutes during the oxidation of xanthine. The corresponding data were recorded for the oxidation of xanthine by XO in the presence of 10 μM NFA over a period of 0-10 minutes (see FIG. 2B).

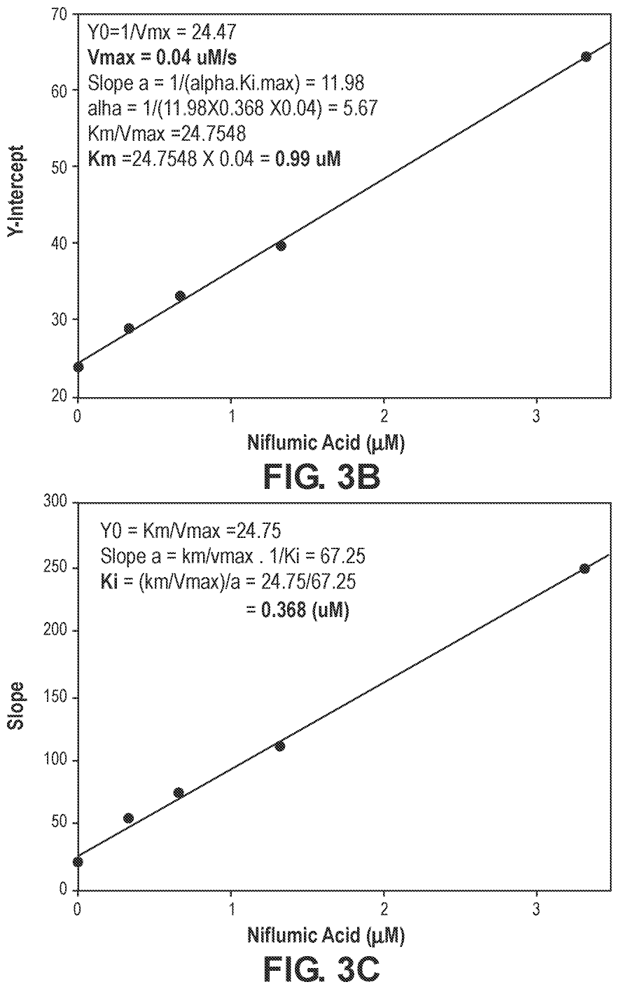

Enzyme kinetics analysis of NFA—The inhibition kinetics of NFA on XO was determined by using the Lineweaver-Burk plot (see FIG. 3A). The Lineweaver-Burk plot showed that a group of lines passed through the same Y-intercept in the second quadrant, indicating a mixed-type inhibition with an inhibitor constant (Kᵢ) determined to be 0.368 μM. Thus, NFA interacted with both the free enzyme and the enzyme-substrate intermediate. The maximum velocity (Vₘₐₓ) and Michaelis-Menten constant of XO were determined in the presence of different concentrations of NFA (see FIG. 3B). The Vₘₐₓ and Kₘ of XO were determined to be 0.04 μM/s and 0.99 μM, respectively. The inhibitor constant (Kᵢ) of NFA was also determined, using the kinetic data, to be 0.368 μM (see FIG. 3C).

Structure activity relationship of XO inhibition—The inhibition of XO activity by 25 different compounds shown in Scheme 1, including niflumic acid (NFA), was studied.

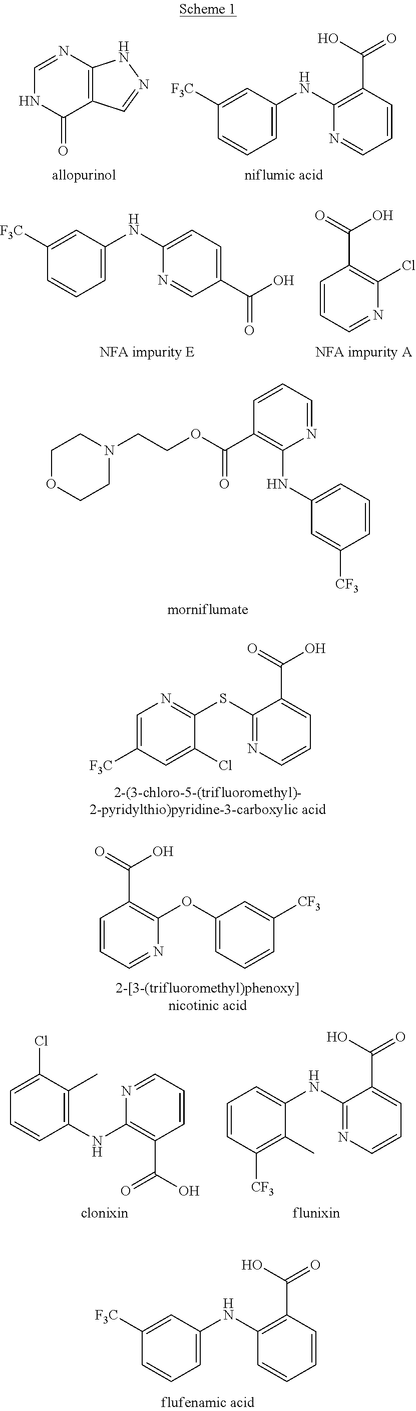

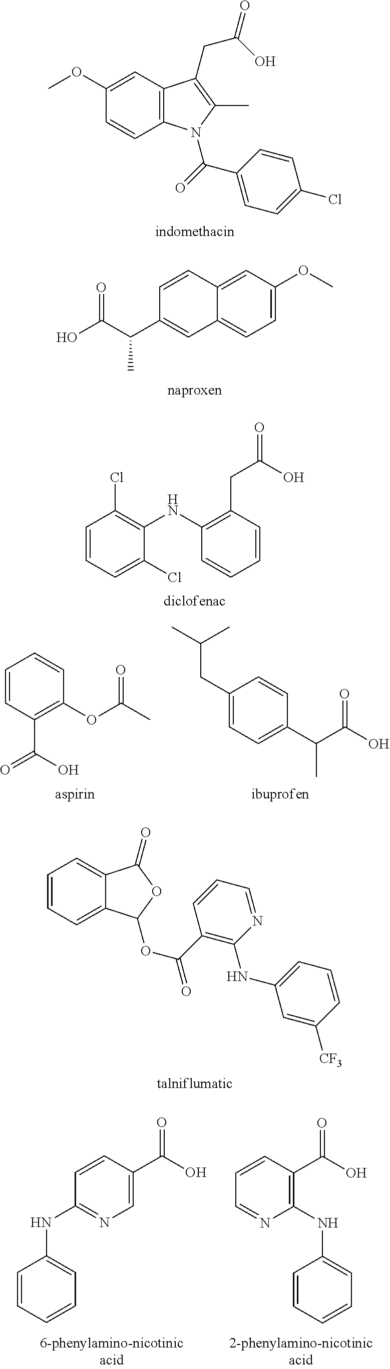

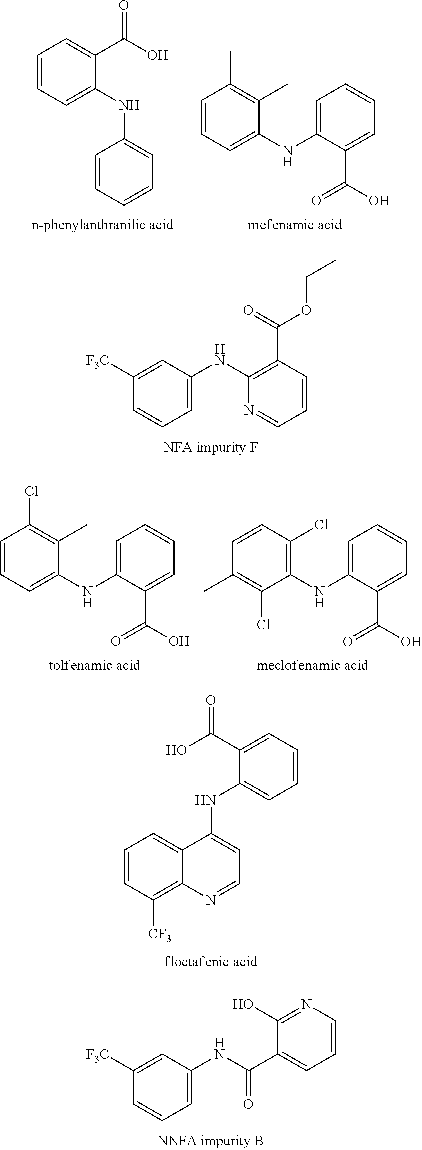

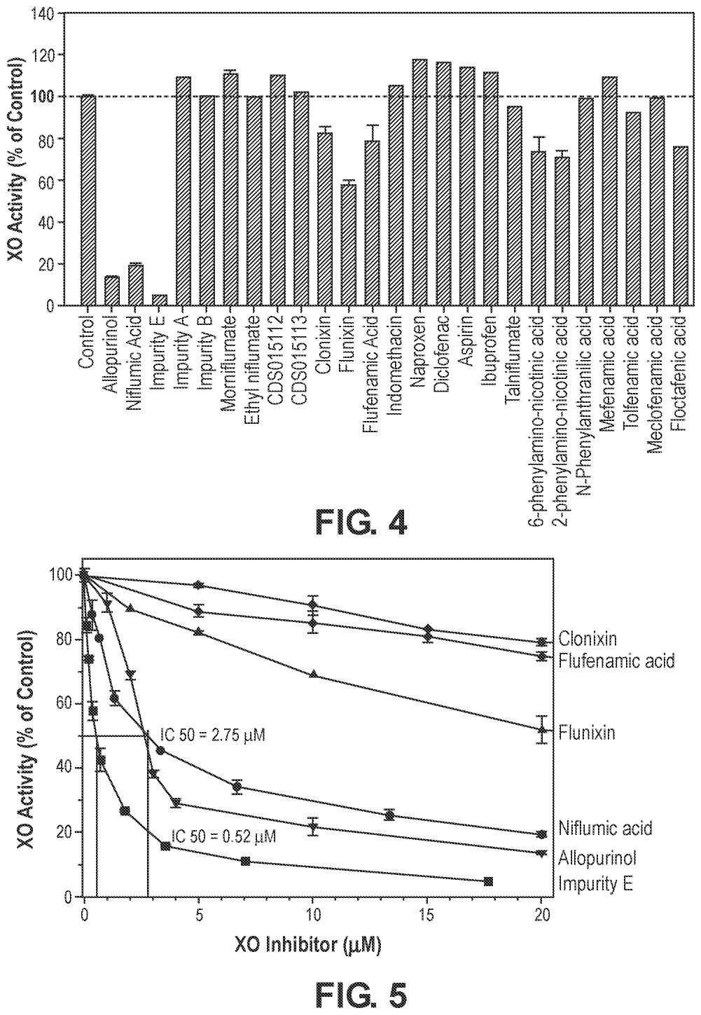

The ability of each compound to inhibit XO at a concentration of 20 μM was compared with that of allopurinol (see FIG. 4). While the compounds share common structural features to that of niflumic acid (NFA), the inhibitory effects on XO varied between the compounds studied NFA had an inhibitory effect similar to that of allopurinol, while NFA impurity E was found to be the most potent inhibitor amongst the compounds tested.

Notably, the two compounds which are further substituted with a trifluoromethyl-anilino moiety at the 2-position (NFA) or 4-position (NFA impurity E) were found to most effectively inhibit XO activity. Although it contains the aforementioned structural features, the methyl group present in flunixin decreased its XO inhibitory activity.

Dose-dependent effects—The dose-dependent effects on XO inhibition by NFA, NFA impurity E (labeled as “Impurity E”), flunixin, clonixin, and flufenamic acid were studied (see FIG. 5). Varying concentrations (0-40 μM) of each compound were pre-incubated with 20 nM of XO. Allopurinol was used as a positive control and DMSO was used as a negative control. NFA impurity E was found to be the most potent amongst each of the compounds tested.

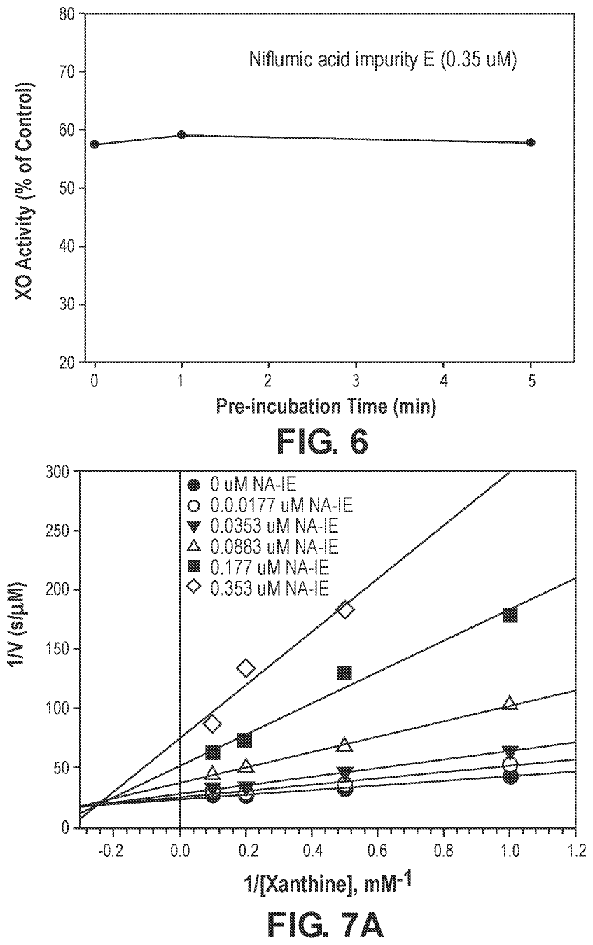

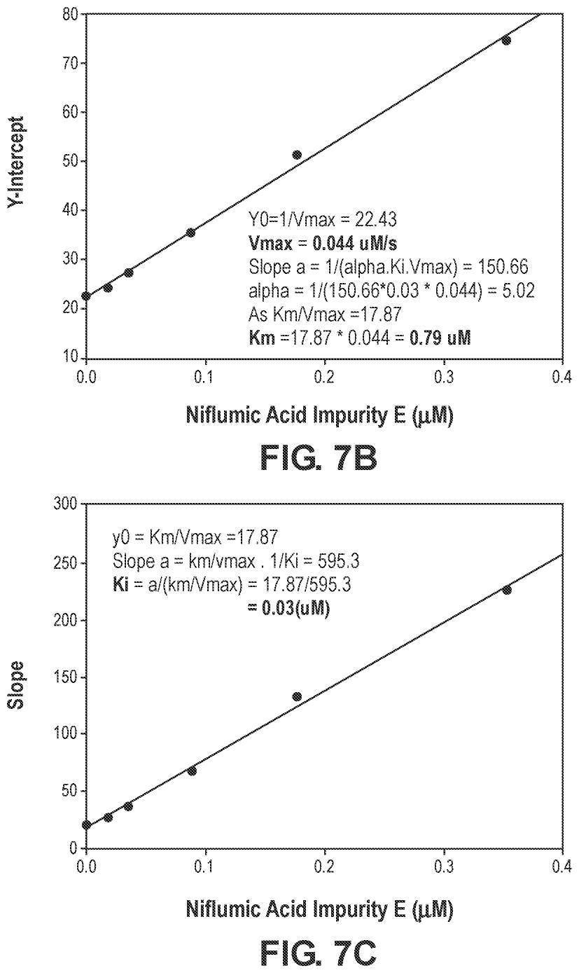

XO inhibition by NFA Impurity E—The influence of pre-incubation on the inhibitory effect of niflumic acid (NFA) impurity E on xanthine oxidase (XO) was determined. When NFA impurity E was pre-incubated with XO for varying amounts of time followed by the addition of xanthine, the initial rate of uric acid formation did not change with longer pre-incubation times (see FIG. 6). The inhibition kinetics of NFA impurity E on XO was determined by using the Lineweaver-Burk plot (see FIG. 7A). The Lineweaver-Burk plot showed that a group of lines passed through the same Y-intercept in the second quadrant, indicating a mixed-type inhibition with an inhibitor constant (Kᵢ) determined to be 0.03 μM. Thus, NFA interacted with both the free enzyme and the enzyme-substrate intermediate. The maximum velocity (Vₘₐₓ) and Michaelis-Menten constant of XO was determined in the presence of different concentrations of NFA impurity F (see FIG. 7B). The Vₘₐₓ and Kₘ of XO were determined to be 0.044 μM/s and 0.79 μM, respectively. The inhibitor constant (Kᵢ) of NFA impurity E was determined to be 0.03 μM from the kinetic data (see FIG. 7C).

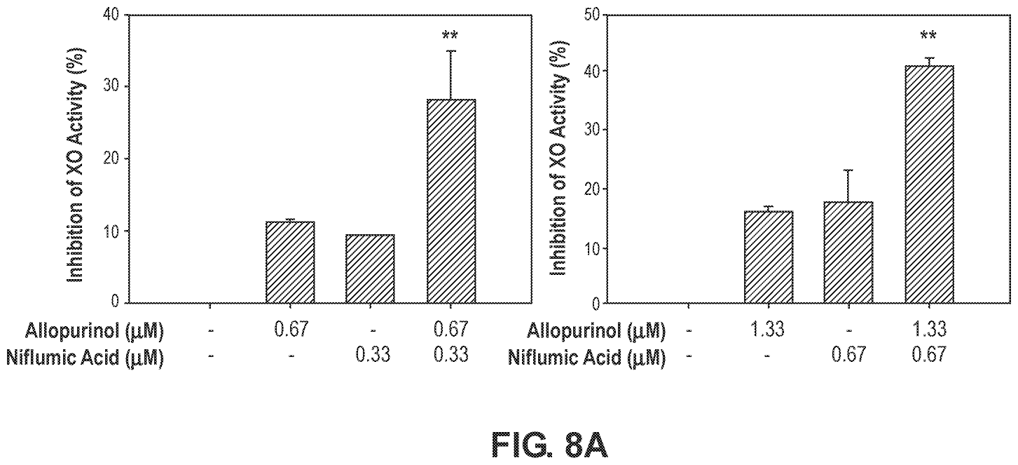

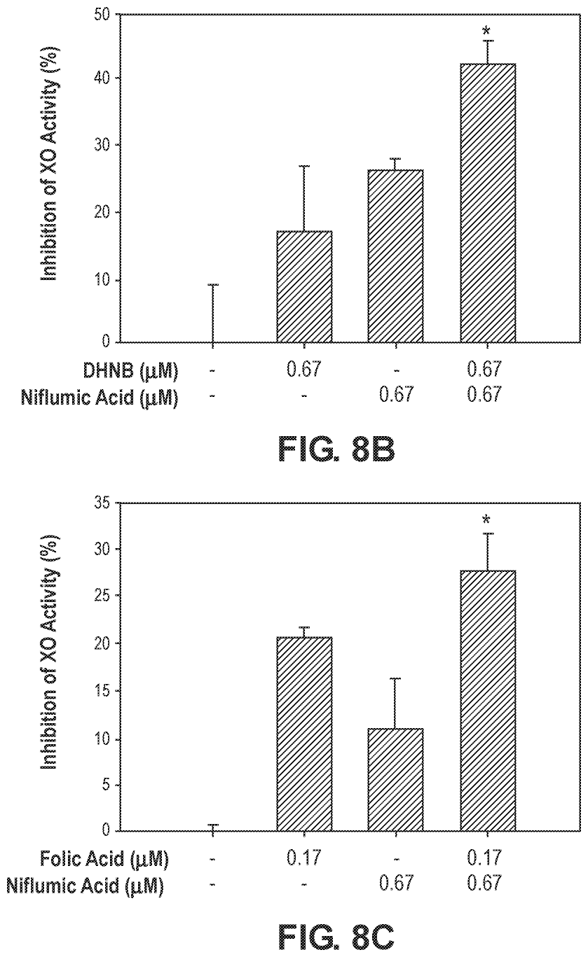

Effects of combinations of different compounds on XO inhibition—To determine whether NFA had an additive or synergistic effect on the inhibition of XO activity with other compounds, NFA was combined, in varying amounts, with allopurinol (see FIG. 8A, left and right panels), 3,4-dihydroxy-5-nitrobenzaldehyde (DHNB) (see FIG. 8B), and folic acid (see FIG. 8C). XO (20 nM) was pre-incubated with NFA, allopurinol or a combination of NFA and allopurinol for 1 minute in a phosphate buffer (100 μM pH 7.2), followed by the addition of 50 mM xanthine to start the reaction. XO activity was analyzed by measuring the production of uric acid. NFA and allopurinol showed a synergistic effect at low concentrations, such as 0.33 μM NFA with 0.67 μM allopurinol (FIG. 8A, left panel), and 0.67 μM NFA with 1.33 μM allopurinol (FIG. 8A, right panel). A combination of NFA (0.67 uM) and DHNB (0.67 uM) had an additive effect on the inhibition of XO activity. A combination of NFA (0.67 uM) and folic acid (0.17 uM) had an additive effect on the inhibition of XO activity.

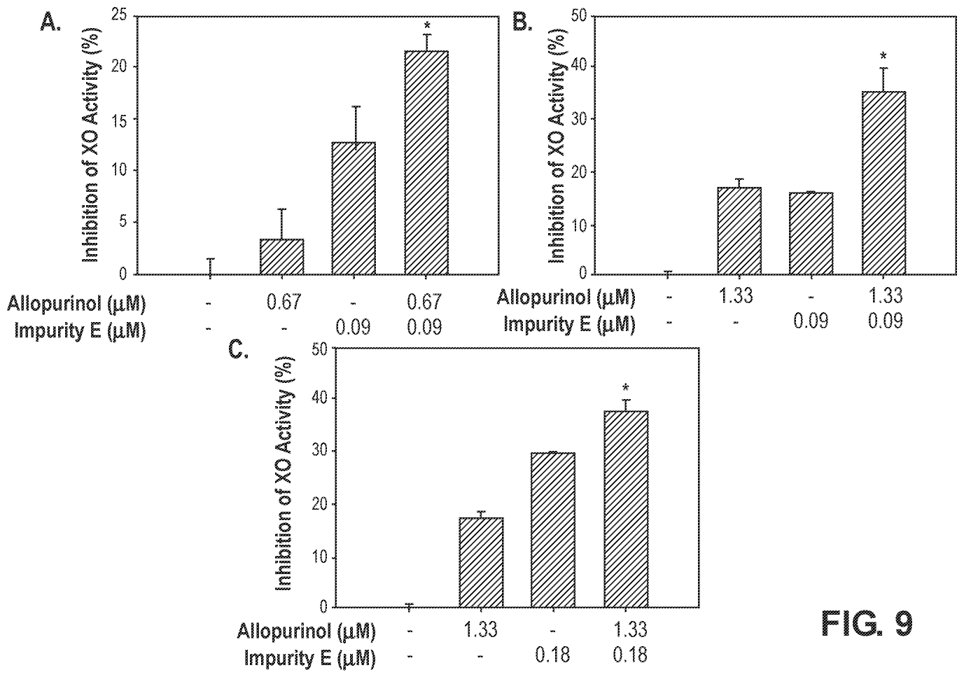

An analogous study was performed to determine the effect on the inhibition of XO activity when NFA impurity E was combined with allopurinol. The concentrations of both compounds were varied over three separate trials (see FIG. 9). Combinations of NFA impurity E and allopurinol in all measured concentrations exhibited an additive effect on the inhibition of XO activity.

### Example 2: Serum Uric Acid Reduction in a Chronic Hyperuricemia Model

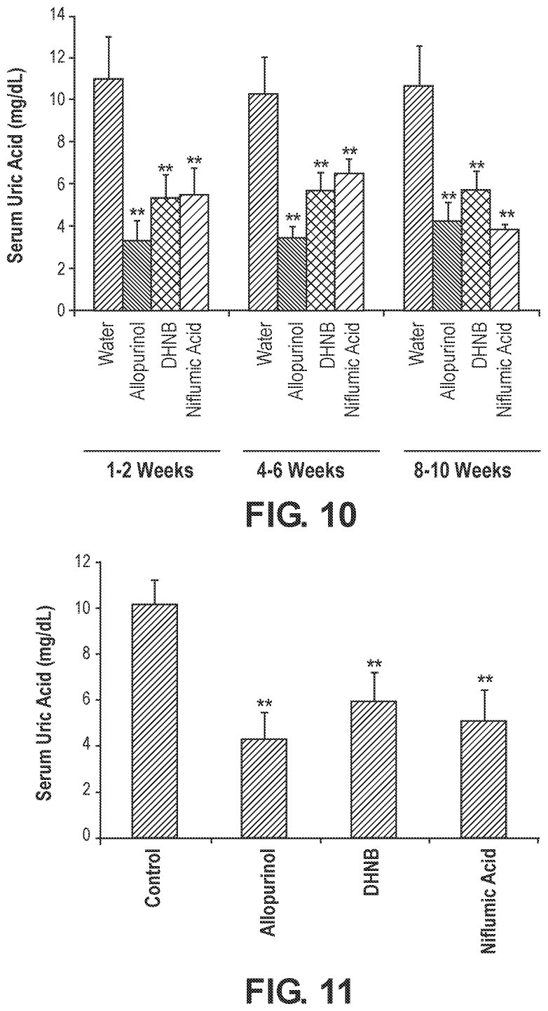

Serum uric acid (SUA) levels in uricase −/− mice were used to probe the effects of treatment with drinking water containing allopurinol, DHNB, and niflumic acid (NFA). Under the maintenance of allopurinol, adult uricase−/− mice show relatively low SUA (2-4 mg/dL). However, once allopurinol is discontinued, uricase−/− mice will exhibit high SUA levels (6-10 mg/dL) in one week. For the current experiments, the allopurinol water was stopped and normal water was supplied to uricase−/− mice for one week; and then uricase−/− mice were treated with DHNB (150 mg/L) or NFA (150 mg/L) in drinking water. Additionally, uricase−/− mice were given allopurinol (150 mg/L) as a positive control, while normal water was given for a negative control. All drug/waters were refreshed every week. Serum uric acid (SUA) levels of treated and control mice were determined 1-2 weeks, 4-6 weeks or 8-10 weeks after the treatment (see FIG. 10). Mouse blood (100˜200 μL) was taken via facial vein at the scheduled time point. The blood was allowed to clot for 1 hour at room temperature and then centrifuged at 2350 g for 4 minutes to obtain serum. The serum was kept on ice and assayed immediately by the tungsten phosphate method, as known to those of skill in the art. The results of the treatment with NFA, DFNB, or allopurinol, after the indicated periods, are summarized in Tables 1A-C. The results of the treatment indicate that NFA significantly reduced SUA levels of uricase−/− mice as compared to that of control mice, which received normal drinking water at all three time points (p<0.001).

TABLE 1A

| Treatment | 1-2 weeks | | |
|---|---|---|---|
| (150 mg/L) | SUA (mg/dl) | Animal # (n) | T-test (p value) |
| Water (Control) | 10.96 ± 1.97 | 5 | |
| Allopurinol | 3.34 ± 0.87 | 9 | 0.0002 |
| DHNB | 5.37 ± 1.06 | 12 | 0.0009 |
| Niflumic acid | 5.47 ± 1.21 | 7 | 0.0006 |

TABLE 1B

| Treatment | 4-6 weeks | | |
|---|---|---|---|
| (150 mg/L) | SUA (mg/dl) | Animal # (n) | T-test (p value) |
| Water (Control) | 10.29 ± 1.69 | 12 | |
| Allopurinol | 3.45 ± 0.49 | 9 | 2.23E−09 |
| DHNB | 5.73 ± 0.76 | 18 | 2.33E−07 |
| Niflumic acid | 6.50 ± 0.64 | 12 | 2.02E−06 |

TABLE 1C

| Treatment | 8-10 weeks | | |
|---|---|---|---|
| (150 mg/L) | SUA (mg/dl) | Animal # (n) | T-test (p value) |
| Water (Control) | 10.66 ± 1.81 | 19 | |
| Allopurinol | 4.28 ± 0.80 | 10 | 2.17E−13 |
| DHNB | 5.76 ± 0.81 | 8 | 3.31E−10 |
| Niflumic acid | 3.89 ± 0.20 | 8 | 7.87E−13 |

An analogous study was performed to investigate the effects on SUA in uricase −/− mice treated with solid chow diet formulated with NFA, DHNB, and allopurinol. NFA, DHNB or allopurinol was formulated into solid chow diet (250 mg/kg food, LabDiet® 5V5R, ½″ pellet; Irradiated) from the company (Test Diet, St. Louis, MO and Lab Supply, Inc). Allopurinol water was stopped for uricase−/− mice, while normal water was supplied during experiments. Uricase−/− mice (1-2 months age, male and female), were given special solid chow die including NFA, DHNB or allopurinol (250 mg/kg food). In separate mice, special solid chow diet containing allopurinol (250 mg/kg food) was given for a positive control and regular solid chow diet was given for a negative control. Blood samples were obtained and processed at 6 or 8 weeks after the treatment (see FIG. 11). Serum uric acid was determined using the tungsten phosphate method. As summarized in Table 2, NFA, DHNB or allopurinol-containing solid chow diet for 6 to 8 weeks significantly reduced SUA levels of uricase−/− mice as regular solid chow diet control group (p<0.001).

TABLE 2

| Treatment | 6-8 weeks | | |
|---|---|---|---|
| (250 mg/Kg food) | SUA (mg/dl) | Animal # (n) | T-test (p value) |
| Regular food (Control) | 10.21 ± 0.97 | 5 | |
| Allopurinol | 4.33 ± 1.15 | 10 | 9.45E−11 |
| DHNB | 5.91 ± 1.30 | 15 | 6.89E−11 |
| Niflumic acid | 5.17 ± 1.28 | 12 | 1.9E−10 |

### Example 3: Hypouricemic Effect of Mornifluraaate in Allantoxanamide Induced Hyperuricemic Mice

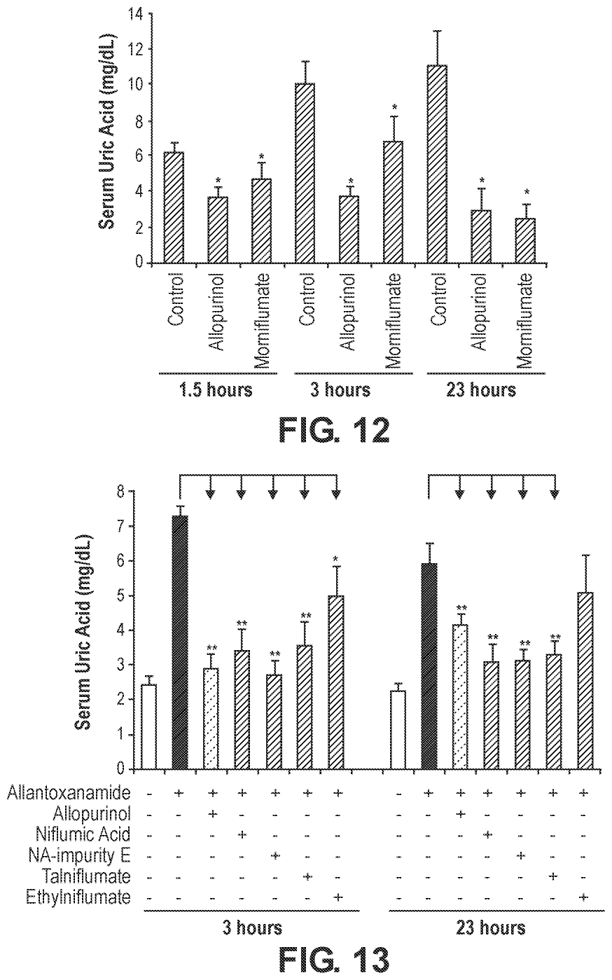

A hyperuricemia mouse model was used. Allantoxanamide, a potent uricase inhibitor, was used to induce hyperuricemia in mice in this study. Briefly, adult C57BL/6 mice (15-25 g. 6-8 weeks old) were injected intraperitoneally (i.p.) with allantoxanamide at 100 mg/kg or 200 mg/kg in 0.5% sodium carboxymethyl cellulose (CMC-Na) in a volume of 0.1 ml/10 g mouse body weight, to increase the serum uric acid level. The mice then received the test compound morniflumate at a concentration of 50 mg/kg in 1.0% polyethylene glycol 400 (PEG400 in a volume of 0.1 ml/10 g mouse body weight), via oral gavage. Positive control mice received allopurinol at the same concentration as test compound after i.p. injection of allantoxanamide. The negative control mice received PEG400 only after i.p, injection of allantoxanamide. Blood samples were collected through the facial vein at 1,5 hours, 3 hours, and 23 hours after the oral gavage. The blood was allowed to clot for 1 hour at room temperature and then centrifuged at 2350×g for 4 minutes to obtain serum. The serum was kept on ice and assayed immediately. Serum uric acid was determined using the phosphotungstate method, as known to those of skill in the art. Allantoxanamide-treated mice showed a progressive increase of SUA levels over the course of the study. One time treatment of morniflumate at a dose of 50 mg/kg significantly reduced SUA levels in allantoxanamide-treated mice at all three time points (1.5 hours, 3 hours, and 23 hours after the treatment of momfilumate) (p<0.05). As a positive control, allopurinol at the same dose also significantly reduced SUA levels in allantoxanamide-treated mice at all three time points (p<0.05). See FIG. 12.

### Example 4: Compounds Effectively Reduce Serum Uric Add (SEA) levels in an Acute Hyperuricemia Mouse Model (Allautoxannamide-Treated Mouse Model)

Adult C57BL/6 mice (15-25 g, 6-8 weeks old) were injected intraperitoneally (i.p.) with allantoxanamide at 100 mg/kg in 0.5% sodium carboxymethyl cellulose (CMC-Na) in a volume of 0.1 ml/10 g mouse body weight, to increase the serum uric acid level. Then the mice received a test compound at a concentration of 50 mg/kg in 1.0% polyethylene glycol 400 (PEG-400 in a volume of 0.1 ml/10 g mouse body weight), via oral gavage. Positive control mice received allopurinol at the same concentration as the test compound after i.p. injection of allantoxanamide. The negative control mice received PEG400 only after i.p. injection of allantoxanamide. Blood samples were collected through facial vein at 3 hours and 23 hours after the oral gavage (one time treatment). The blood was allowed to clot for 1 hour at room temperature and then centrifuged at 2350×g for 4 minutes to obtain serum. The serum was kept on ice and assayed immediately. Serum uric acid was determined using the phosphotungstate method, as known to those of skill in the art.

FIG. 13 shows the effect of 4 test compounds (niflumic acid, NFA-impurity E, talniflumate, and ethylniflumate) on serum uric acid levels in allantoxanamide-treated mice. Allopurinol was used as a positive control. Allantoxanamide is a potent uricase inhibitor. In the current study, allantoxanamide (100 mg/kg)-treated mice showed substantial increase of SUA levels. One time treatment of all four compounds at a dose of 50 mg/kg significantly reduced SUA levels in allantoxanamide-treated mice at the 3-hour time point (**P<0.01; *P<0.05) and three compounds (niflumic acid, NFA-impurity E, and talniflumate) also still showed a significant effect at the 23-hour time point after the treatment (**P<0.01). As a positive control, allopurinol at the same dose (50 mg/kg) also significantly reduced SUA levels in allantoxanamide-treated mice at all two time points (p<0.05). Animal numbers used in this experiments [Negative control (baseline)=11; allantoxanamide=10, allopurinol (positive control)=4; niflumic acid=6; NFA-impurity E=6; talniflumate=6 and ethylniflutnate=6].

The compounds and methods of the appended claims are not limited in scope by the specific compounds and methods described herein, which are intended as illustrations of a few aspects of the claims and any compounds and methods that are functionally equivalent are within the scope of this disclosure. Various modifications of the compounds and methods in addition to those shown and described herein are intended to fall within the scope of the appended claims. Further, while only certain representative compounds, methods, and aspects of these compounds and methods are specifically described, other compounds and methods are intended to fall within the scope of the appended claims. Thus, a combination of steps, elements, components, or constituents can be explicitly mentioned herein; however, all other combinations of steps, elements, components, and constituents are included, even though not explicitly stated.

1. A method for treating a disease or condition associated with elevated serum uric acid levels in a subject, comprising:
administering to the subject an effective amount of a pharmaceutical composition comprising a compound of the following formula: 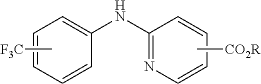
or a pharmaceutically acceptable salt thereof, wherein:
R is hydrogen, substituted or unsubstituted alkyl, substituted or unsubstituted alkenyl, substituted or unsubstituted alkynyl, substituted or unsubstituted aryl, or substituted or unsubstituted cycloalkyl.

2. The method of claim 1, wherein the compound is selected from the group consisting of:

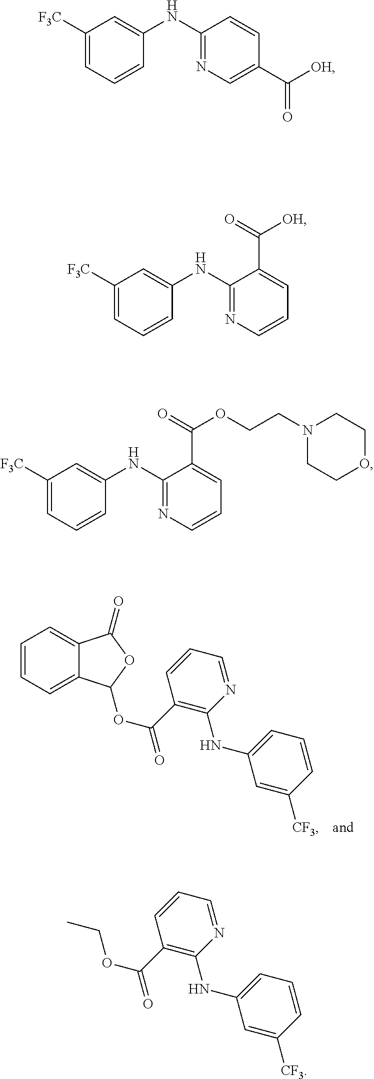 [SMILES: O=C(O)c1ccc(Nc2cccc(C(F)(F)F)c2)nc1 ; O=C(O)c1cccnc1Nc1cccc(C(F)(F)F)c1 ; O=C(OCCN1CCOCC1)c1cccnc1Nc1cccc(C(F)(F)F)c1 ; O=C1OC(OC(=O)c2cccnc2Nc2cccc(C(F)(F)F)c2)c2ccccc21 ; CCOC(=O)c1cccnc1Nc1cccc(C(F)(F)F)c1]

3. The method of claim 1, wherein the disease or condition associated with elevated serum uric acid levels is gout hyperuricemia, or cardiovascular disease.

4. (canceled)

5. The method of claim 1, further comprising administering a second therapeutic agent to the subject.

6. The method of claim 5, wherein the second therapeutic agent comprises allopurinol, folic acid, or 3,4-dihydroxy-5-nitrobenzaldehyde.

7. The method of claim 1, further comprising selecting a subject having a disease or condition associated with elevated serum uric acid levels.

8. The method of claim 1, wherein the pharmaceutical composition is substantially free from tetrazoles, triazoles, or solubility enhancers for monosodium urate.

9. A method of reducing serum uric acid levels in a subject, comprising:
administering to the subject an effective amount of a pharmaceutical composition comprising a compound of the following formula: 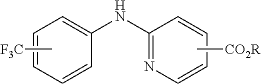
or a pharmaceutically acceptable salt thereof, wherein:
R is hydrogen, substituted or unsubstituted alkyl, substituted or unsubstituted alkenyl, substituted or unsubstituted alkynyl, substituted or unsubstituted aryl, or substituted or unsubstituted cycloalkyl.

10. The method of claim 9, wherein the compound is selected from the group consisting of:

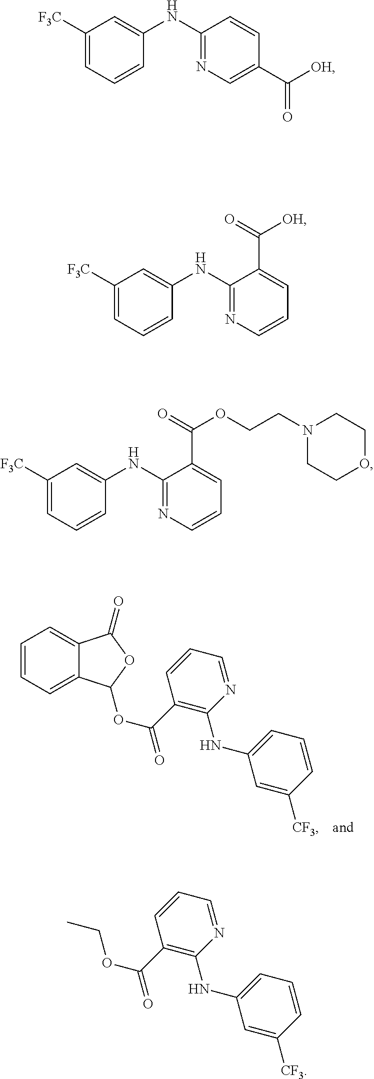 [SMILES: O=C(O)c1ccc(Nc2cccc(C(F)(F)F)c2)nc1 ; O=C(O)c1cccnc1Nc1cccc(C(F)(F)F)c1 ; O=C(OCCN1CCOCC1)c1cccnc1Nc1cccc(C(F)(F)F)c1 ; O=C1OC(OC(=O)c2cccnc2Nc2cccc(C(F)(F)F)c2)c2ccccc21 ; CCOC(=O)c1cccnc1Nc1cccc(C(F)(F)F)c1]

11. The method of claim 9, further comprising selecting a subject having a disease or condition associated with elevated serum uric acid levels.

12. The method of claim 11, wherein the disease or condition associated with elevated serum uric acid levels is gout hyperuricemia, or cardiovascular disease.

13. (canceled)

14. The method of claim 9, further comprising administering a second therapeutic agent to the subject.

15. The method of claim 14, wherein the second therapeutic agent comprises allopurinol, folic acid, or 3,4-dihydroxy-5-nitrobenzaldehyde.

16. The method of claim 9, wherein the pharmaceutical composition is substantially free from tetrazoles, triazoles, or solubility enhancers for monosodium urate.

17. A method of inhibiting xanthine oxidase activity in a cell, comprising:
contacting a cell with an effective amount of a composition comprising a compound of the following formula: 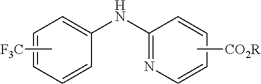
or a pharmaceutically acceptable salt thereof, wherein:
R is hydrogen, substituted or unsubstituted alkyl, substituted or unsubstituted alkenyl, substituted or unsubstituted alkynyl, substituted or unsubstituted aryl, or substituted or unsubstituted cycloalkyl.

18. The method of claim 17, wherein the compound is selected from the group consisting of:

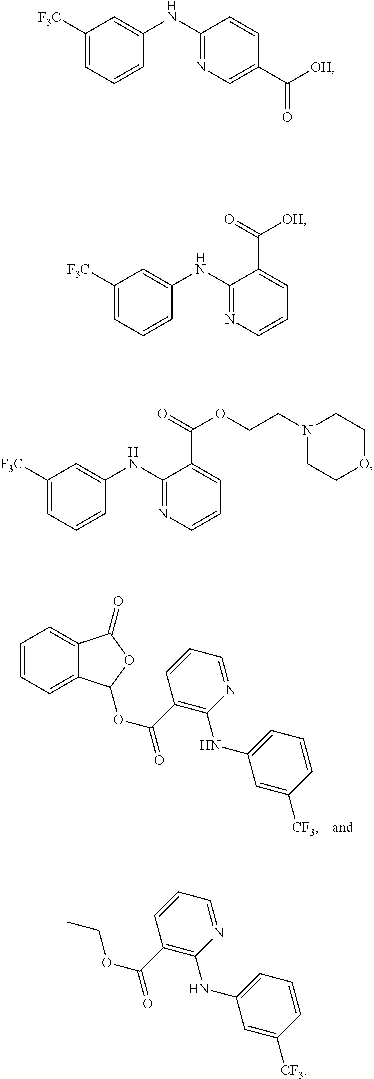 [SMILES: O=C(O)c1ccc(Nc2cccc(C(F)(F)F)c2)nc1 ; O=C(O)c1cccnc1Nc1cccc(C(F)(F)F)c1 ; O=C(OCCN1CCOCC1)c1cccnc1Nc1cccc(C(F)(F)F)c1 ; O=C1OC(OC(=O)c2cccnc2Nc2cccc(C(F)(F)F)c2)c2ccccc21 ; CCOC(=O)c1cccnc1Nc1cccc(C(F)(F)F)c1]

19. The method of claim 17, wherein the contacting is performed in vivo or in vitro.

20. (canceled)

21. The method of claim 17, wherein the composition is substantially free from tetrazoles, triazoles, or solubility enhancers for monosodium urate.

22. A pharmaceutical composition, comprising: 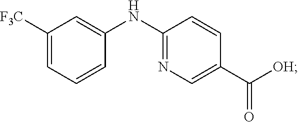 [SMILES: O=C(O)c1ccc(Nc2cccc(C(F)(F)F)c2)nc1]
and a pharmaceutically acceptable carrier.

23. The pharmaceutical composition of claim 22, further comprising a xanthine oxidase inhibitor.

24. The pharmaceutical composition of claim 23, wherein the xanthine oxidase inhibitor comprises allopurinol.

25. The pharmaceutical composition of claim 22, wherein the pharmaceutical composition is substantially free from tetrazoles, triazoles, or solubility enhancers for monosodium urate.
